(19) United States
(12) Patent Application Publication
(10) Pub. No.: US 2011/0262356 A1
(43) Pub. Date: Oct. 27, 2011

(54) Active Particles for Bio-Analytical Applications and Methods for Preparation Thereof

(75) Inventors: Sara Bonacchi, Pistoia (IT); Ricarrdo Juris, Bologna (IT); Marco Montalti, Calderara Di Reno (IT); Luca Prodi, Bologna (IT); Enrico Rampazzo, Bologna (IT); Nelsi Zaccheroni, Bologna (IT); Leopoldo Della Ciana, Bologna (IT); Serena Fabbroni, Medicina (IT); Stafano Grilli, Medicina (IT); Ettore Marzocchi, Bologna (IT)

(73) Assignees: CYANAGEN S.R.L., Bologna (IT); ALMA MASTER STUDIORUM-UNIVERSITA' DI BOLOGNA, Bologna (IT)

(21) Appl. No.: 13/056,700

(22) PCT Filed: Jul. 31, 2009
(86) PCT No.: PCT/IB09/06435

§ 371 Date: Jun. 28, 2011

(30) Foreign Application Priority Data
Jul. 31, 2008 (IT) BO2008A000487

(51) Int. Cl. A61K 49/00; A61K 9/14; A61K 31/352; A61K 31/403; A61K 31/555; A61K 31/351; A61K 31/695; A61K 31/538; B82Y 5/00

(52) U.S. Cl. 424/9.1; 514/63; 424/400; 514/454; 514/229.8; 514/411; 514/187; 514/459; 977/773; 977/915; 977/927

(57) ABSTRACT

Luminescent and/or electroactive nanoparticles suitable for MRI (Magnetic Resonance Imaging) and/or PET (Positron Emission Tomography) are prepared by mixing luminescent or electroactive compounds and ethyl oxide/propyl oxide block-copolymers in an organic solvent, which is thereafter evaporated in order to obtain a residue; and by hydrolyzing-condensating tetraalkoxysilanes in an aqueous solution in the presence of the residue; the obtained nanoparticles show no or a negligible release of the luminescent or electroactive compounds and are useful for bio-analytic and bio-medic applications.

### TECHNICAL FIELD

The present invention relates to methods for the preparation of an active particle, active particles and uses of these active particles.

### STATE OF THE ART

In the field of bioanalytics, it is currently deeply felt the need to identify new diagnostic tools and in particular particles which can used in applications related to the detection, the labeling of bio-molecules and imaging. It is also currently felt the need to provide new products for phototherapeutic treatments.

In the state of the art, particles are known which can be used for the release of drugs. For example, A New Class of Silica Cross-Linked Micellar Core-Shell Nanoparticles (Huo, Q.; Liu, J.; Wang, L. Q.; Jiang, Y.; Lambert, T. N.; Fang, E. J. Am. Chem. Soc. 2006, 128 (19), 6447-6453) describes a method for the preparation of particles with a silicate core and subsequent loading of a drug.

These types of particles, however, are unlikely to be used for diagnostic purposes as they inherently have a relatively high tendency to release the pharmaceutical active compounds, which are contained in them. The methods for preparing these particles are also often relatively long.

From the foregoing, it appears that there is still a great need to provide new active particles and new methods for their preparation.

### OBJECT OF THE INVENTION

The purpose of this invention is to provide active particles, uses of particles and methods for the preparation of particles, which allow overcoming, at least partially, the drawbacks of the state of the art and are, at the same time, easy and economical to implement.

According to the present invention the following are provided: active particles, uses of particles and methods for the preparation of particles as specified in the independent claims which follow and, preferably, in any of the claims directly or indirectly dependent on the independent claims.

Unless explicitly specified otherwise, the following terms have the meanings indicated below.

By active compound (or particle) is meant a compound (or particle), particularly organic or metallo-organic, which is an emitter and/or electroactive and/or useful for contrast and/or is a positron emitter.

By emitter compound (or particle) is meant a compound (or particle) that can emit energy, preferably in the form of detectable electromagnetic radiations (luminescent compound or particle), or heat. The emitter compound may be able to emit alone and/or in combination with at least one second emitter compound; also by means appropriate energy transfer processes between luminescent species; the emission can occur by fluorescence, phosphorescence, electrochemiluminescence (ECL) processes or chemiluminescent reactions.

An emitter compound may be fluorescent or luminescent. A luminescent compound, in particular, is either phosphorescent or electrochemiluminescent.

By electrochemiluminescent compound is meant a compound, which, when involved in a redox process is capable of emitting detectable electromagnetic radiation.

By electroactive species (compound or particles) are meant chemical species capable of participating in redox processes usable for analytical purposes, for detection, or participating in energy transfer processes with other luminescent species.

By contrast species (compound or particles) are meant species suitable for applications of MRI (magnetic resonance imaging).

By particles are meant corpuscles with an average hydrodynamic diameter in water of less than 500 nm.

By the average hydrodynamic diameter is meant the average diameter of particles as determined in a dispersion of particles in a solvent by means the DLS (dynamic light scattering) technique.

In this text Cₓ-C_{y} is referred to a group with a number of carbon atoms from x to y.

In the present text, by an aliphatic hydrocarbon is meant a non-aromatic and non-substituted hydrocarbon, saturated or unsaturated, linear, branched and/or cyclic. Non-restrictive examples of aliphatic groups are: t-butyl, ethenyl, ethyl, 1- or 2-propenyl, n-propyl, 2-propyl, cyclohexyl, cyclohexenyl.

In the present text, the term alkyl means a saturated aliphatic group (i.e., an aliphatic group with no double or triple carbon-carbon bonds). Non-restrictive examples of alkyls are methyl, ethyl, n-propyl, t-butyl, cyclohexyl.

In this text, the term alkoxy means an aliphatic group (preferably a C₁-C₅ aliphatic group, advantageously a C₁-C₄ alkyl group) linked to the remainder of the molecule through an oxygen atom. Non-restrictive examples of alkoxy groups are: methoxy, ethoxy.

By alkoxy-silane functionality is meant a molecular portion with the Si—O—R^{a} structure, where R^{a} indicates a C₁-C₄ alkyl group, advantageously a C₁-C₂ alkyl group, in particular an ethyl group.

By trialkoxysilane is meant a molecule possessing three alkoxy-silane functionalities, in which the three alkoxy groups of the alkoxy-silane functionalities are connected to the same silicon atom.

By tetraalkoxysilane is meant a molecule having four alkoxy-silane functionalities, in which the four alkoxy groups of the alkoxy-silanes functionalities are connected to the same silicon atom. Tetraethoxysilane (TEOS) is an example of a tetraalkoxysilane.

By substantially hydrophilic chain is meant a chain with water solubility greater than the solubility of a substantially hydrophobic chain. Advantageously, the substantially hydrophilic chain has a higher solubility in water than in ethanol.

By substantially hydrophobic chain is meant a chain that has water solubility lower than the solubility of a substantially hydrophilic chain. Advantageously, the substantially hydrophobic chain is substantially lipophilic.

By substantially lipophilic molecular portion (or chain or compound) is meant a molecular portion (or chain or compound) which has greater solubility in ethanol than in water.

By silanization is meant the carrying out of a process of hydrolysis-condensation where at least part of the alkoxy-silane functionalities are hydrolysed to silanols and where, through condensation reactions, takes place the formation of bridging siloxane bonds (i.e., Si—O—Si), which, advantageously, leads to the formation of a lattice. For the mere purpose of an example, FIG. 1 illustrates schematically the reactions that take place when TEOS (tetraethylorthosilicate or tetraethoxysilane) silanizes.

For aqueous solution is a meant a solution in which the solvent is mostly water. Advantageously, in an aqueous solution the only solvent is water.

Unless explicitly stated otherwise, the content of the references (articles, texts, patent applications, etc.) cited in this text is herein referred to in full for the sake of completeness of description. In particular, the mentioned references are herein incorporated by reference.

### BRIEF DESCRIPTION OF THE DRAWINGS

The invention will now be described with reference to the attached drawings, which illustrate some non-limiting examples of implementation, where:

FIG. 1 schematically illustrates the reactions that take place during the silanization of TEOS;

FIG. 2 shows the absorption spectrum (solid line) and the fluorescence emission spectrum (dashed line) of 8-oxo-3-propylamino-8H-acenaphtho[1,2-b]pyrrole-9-carbonitrile;

FIG. 3 shows the absorption spectrum (solid line) and fluorescence emission spectrum (dashed line) of particles containing 8-oxo-3-propylaminotriethoxyisilyl-8H-acenaphtho[1,2-b]pyrrole-9-carbonitrile;

FIG. 4 illustrates the size distribution obtained by the DLS (dynamic light scattering) technique of particles comprising 8-oxo-3-propylaminotriethoxyisilyl-8H-acenaphtho[1,2-b]pyrrole-9-carbonitrile in water (the abscissa is given the diameter expressed in nm;

FIG. 5 shows the absorption spectrum (solid line) and fluorescence emission spectrum (dashed line) of cyanine CY7ClBIEt in ethanol;

FIG. 6 shows the absorption spectrum (solid line) and fluorescence emission spectrum (dashed line) of particles containing cyanine CY7ClBIEt in water;

FIG. 7 shows the size distribution obtained by the DLS technique (dynamic light scattering) of particles comprising cyanine CY7ClBIEt in water (abscissa shows the diameter expressed in nm);

FIG. 8 illustrates the absorption spectrum (solid line) and fluorescence emission spectrum (dashed line) of 4-(dicyanomethylene)-2-methyl-6-(4-dimethylaminostyryl)-4H-pyran in ethanol;

FIG. 9 shows the absorption spectrum (solid line) and fluorescence emission spectrum (dashed line) of particles comprising 4-(dicyanomethylene)-2-methyl-6-(4-dimethylaminostyryl)-4H-pyran in water;

FIG. 10 shows the size distribution obtained by the DLS (dynamic light scattering) technique of particles comprising 4-(dicyanomethylene)-2-methyl-6-(4-dimethylaminostyryl)-4H-pyran in water;

FIG. 11 illustrates the absorption spectrum (solid line) and luminescence emission spectrum (dotted line) of the complex (acetylacetonato)bis[2-phenylpyridinato-C², N]iridium (III); Ir (III)(pq)₂ acac in ethanol;

FIG. 12 shows the absorption spectrum (solid line) and luminescence emission spectrum (dashed line) of particles containing the complex (acetylacetonato)bis[2-phenylpyridinato-C², N]iridium (III); Ir (III)(pq)₂ acac in water;

FIG. 13 illustrates the size distribution obtained by the DLS (dynamic light scattering) technique of particles comprising the complex (acetylacetonato)bis[2-phenylpyridinato-C²,N]iridium (III), Ir(III)(pq)₂acac in water;

FIG. 14 illustrates the absorbance of particles in accordance with the invention (columns 3 and 4) and comparison particles (columns 1 and 2);

FIG. 15 illustrates the absorbance of particles in accordance with the invention (columns 3 and 4) and comparison particles (columns 1 and 2);

FIG. 16 shows three images acquired during in vivo imaging of athymic mice nu/nu with particles in accordance with the invention, comprising the cyanine CY7ClBIEt (to 0.1% by moles vs. the moles of TEOS);

FIG. 17 shows three images acquired during in vivo imaging of athymic mice nu/nu with commercial luminescent particles (Invitrogen QDs 800); and

FIG. 18 illustrates schematically and for merely exemplificative purposes a method in accordance with the present invention.

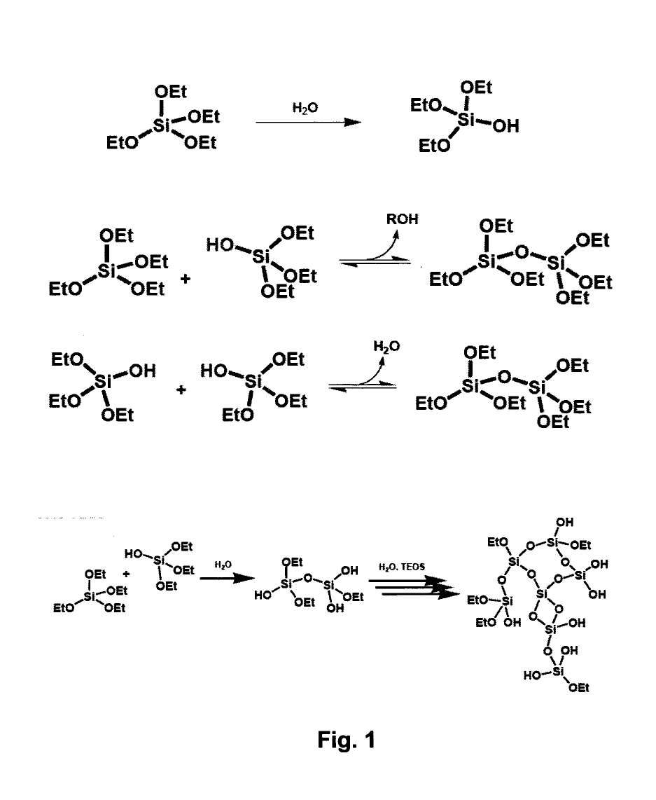

### EMBODIMENTS OF THE INVENTION

In accordance with a first aspect of the present invention, it is provided a method for the preparation of an active nanoparticle, comprising a mixing step, during which at least one active compound is mixed with molecules of at least one surfactant in an organic solvent; an evaporation step, that is subsequent to the mixing step and during which the organic solvent is evaporated in order to obtain a residue; a reaction step, which is subsequent to the evaporation step and during which molecules of at least one alkoxysilane are added to the residue and silanized in presence of water; the alkoxysilane is chosen between a tetraalkoxysilane and a trialkoxysilane; the surfactant comprising the following structure:

Hydro¹-Lipo-Hydro²

wherein Lipo indicates a substantially hydrophobic chain, Hydro¹ and Hydro² each indicate a substantially hydrophilic chain. Advantageously, the active compound is substantially lipophilic.

FIG. 18 schematically shows, in an exclusively exemplificative and not at all limitative way, the formation of the particles (shown on the right): the molecules of the surfactant (or of surfactants, shown on the left) in the presence of water form micelles (shown in the middle); the alkoxysilane silanizes so as to form a core (shown on the right in the area of a central portion of the particles).

In the present text, by micelles it is meant either micellar aggregates (containing molecules of only one type of surfactants) or micellar co-aggregates (containing molecules of many types of surfactants).

According to some embodiments, micelles are micellar aggregates.

One has to note that, advantageously, the active compound is different from the surfactant. Advantageously, the active compound is different from the alkoxysilane. Advantageously, the alkoxysilane is different from the surfactant.

According to some embodiments, the reaction step takes place at a temperature from 10° C. to 60° C., advantageously from 20° C. to 50° C., advantageously from 25° C. to 40° C. According to some embodiments, the reaction step takes place at a temperature from 10° C. to 80° C., advantageously from 15° C. to 60° C., advantageously from 20° C. to 30° C.

Advantageously, the active compound is an emitter compound. Advantageously, the active compound is luminescent or fluorescent.

The organic solvents that can be used during the mixing step are several. According to some embodiments, the organic solvent is selected in a group consisting of: methanol, chloroform, dichloromethane, tetrahydrofurane, acetonitrile, toluene, ethanol.

According to some embodiments, the active compound is a photoluminescent compound, that is to say a chemical species able to emit detectable electromagnetic radiations, advantageously with wavelengths from 200 nm to 1500 nm, advantageously higher than 500 nm, advantageously from 550 nm to 1500 nm.

According to some embodiments, Lipo is substantially lipophilic. Advantageously, Hydro¹ and Hydro² are more soluble in water than in ethanol.

According to some embodiments, Hydro¹ represents a chain

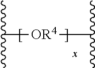

wherein R⁴ is a linear alkyl C₁-C₃ (advantageously C₂-C₃); Hydro² represents a chain

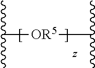

wherein R⁵ is a linear alkyl C₁-C₃ (advantageously C₂-C₃); Lipo represents a chain

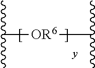

wherein R⁶ is a branched alkyl C₃-C₄.

Advantageously, R⁴ and R⁵ represent, each one independently from the other, an ethyl. Advantageously, R⁶ represent a branched propyl.

Advantageously the surfactant is a block co-polymer ethylene oxide/propylene oxide.

According to some embodiments, y is lower than or equal to x and z; x is from 40 to 130; z is from 40 to 130; y is from 20 to 85. Advantageously, x is from 55 to 130; z is from 55 to 130; y is from 35 to 85. Advantageously, x is from 80 to 120; z is from 80 to 120; y is from 50 to 80. Advantageously, x and z are from 90 to 110 and y is from 60 to 70.

Advantageously, Hydro¹ represents a chain

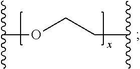

Hydro² represents a chain

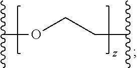

Lipo represents a chain

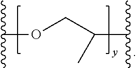

Usually the structure Hydro¹-Lipo-Hydro² has at its left extremity one terminal hydrogen atom bound to oxygen, and at its right extremity one hydroxide. This is exemplified by Pluronic® F127:

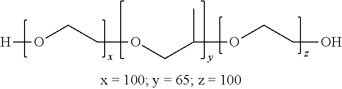

Alternatively, the extremities of the structure Hydro¹-Lipo-Hydro² can be functionalized in different ways.

According to some embodiments, the particle has an average hydrodynamic diameter in water lower than 100 nm, in particular from circa 40 to circa 100 nm.

According to some embodiments, the surfactant has a mean molecular weight of at least 6 KDa, advantageously of at least 10 KDa. In particular, the ratio between the mean molecular weight of Lipo and the mean molecular weight of Hydro¹ and between the mean molecular weight of Lipo and the mean molecular weight of Hydro² are, each independently from one another, from circa 0.4 to circa 2.0.

Advantageously, the surfactant has a mean molecular weight less than 16 KDa. Advantageously, the surfactant has a mean molecular weight lower than 15 KDa. Advantageously, the ratios z/y and x/y are, each, higher than circa 1.3 and lower than circa 1.7.

According to some embodiments, the surfactant is chosen among the group consisting of Pluronic® F127, F98, P105, F68, F108, F88, F87.

According to some embodiments, the alkoxysilane has a formula selected in the group consisting of:

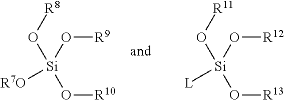

wherein R⁷, R⁸, R⁹, R¹⁰, R¹¹, R¹², and R¹³ represent, independently of each other, an alkyl C₁-C₄; L represents a molecular portion substantially lipophilic. Advantageously, R⁷, R⁸, R⁹, R¹⁰, R¹¹, R¹², and R¹³ are, independently of each other, an alkyl C₁-C₂.

According to some embodiments, L represents an alkyl C₁-C₄. Advantageously, L represents an alkyl C₁-C₂.

Advantageously, the alkoxysilane has the formula:

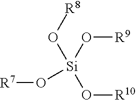

According to some embodiments, the alkoxysilane is chosen in the group consisting of:

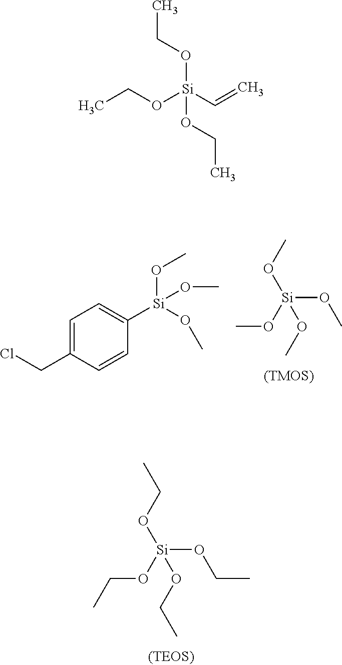

According to some embodiments, the alkoxysilane is chosen among the group consisting of TMOS e TEOS. Advantageously, the alkoxysilane is TEOS.

According to some embodiments, the reaction step takes place in an aqueous solution. In particular, the aqueous solution has pH lower than circa 5, advantageously higher than circa 0, or higher than circa 9, advantageously lower than circa 13. According to some embodiments, the reaction step takes place in an aqueous solution with a pH lower than circa 5, or higher than circa 9; the pH is higher than circa 0.5 and lower than circa 12.

Advantageously, the reaction step takes place in solution; at the beginning of the reaction step, the molar percentage ratio between the active compound and the alkoxysilane is from circa 0.002% to circa 5%, in particular from circa 0.01% to circa 0.5% (more precisely, to circa 0.2%). Advantageously, at the beginning of the reaction step, the molar ratio between the alkoxysilane and the surfactant is lower than or equal to circa 110.

According to some embodiments: when the surfactant has a mean molecular weight higher than circa 10 KDa, the molar ratio between the alkoxysilane and the surfactant is from circa 110 to circa 90. When the surfactant has a mean molecular weight from circa 8 to circa 10 KDa, the molar ratio between the alkoxysilane and the surfactant is from circa 90 to circa 20. When the surfactant has a mean molecular weight from circa 6 to circa 8 KDa, the molar ratio between the alkoxysilane and the surfactant is from circa 20 to circa 9. When the surfactant has a mean molecular weight from circa 3 to circa 6 KDa, the molar ratio between the alkoxysilane and the surfactant is from circa 9 to circa 4.

In the majority of the embodiments, the reaction step lasts less than circa 6 hours. Advantageously, the reaction step lasts more than circa 1 hour, specifically is of circa 1 hour and 45 minutes.

According to some embodiments, the above disclosed method comprises a termination step, during which the reaction is stopped by means of the addition of a termination compound chosen in the group consisting of: monoalkoxysilane, dialkoxysilane, monohalosilane, dihalosilane; in particular, the termination step is subsequent to the reaction step.

Advantageously, the termination compound is chosen among: dialkoxysilane, in particular diethoxydimethylsilane, and monohalosilane, in particular chlorotrimethylsilane.

By dihalosilane is meant a molecule that having a silicon bound to only two halogens, advantageously Cl, Br, I, advantageously Cl. Advantageously, the dihalosilane is chosen in the group consisting of

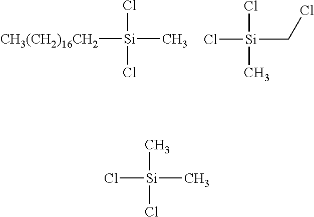

By monohalosilane is meant a molecule that has a silicon bound to only one halogen, advantageously Cl, Br, I, advantageously Cl. Advantageously, the monohalosilane is chosen in the group consisting of:

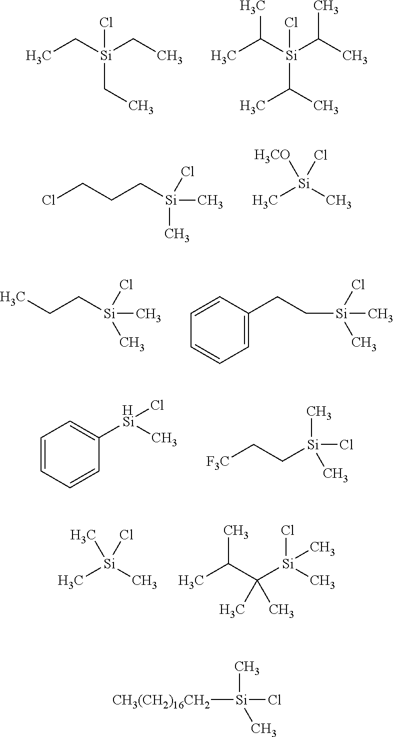

By dialkoxysilane is meant a molecule that has only two alkoxysilane moieties, wherein the two alkoxy groups of the alkoxysilane moieties are bound to the same silicon atom. Advantageously, the dialkoxysilane is chosen among the group consisting of:

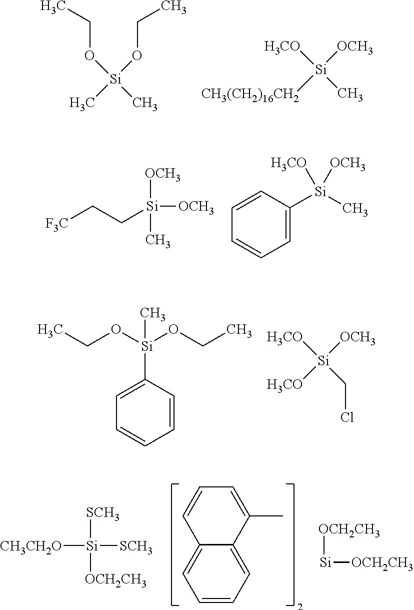

By monoalkoxysilane is meant a molecule that has only one alkoxysilane moiety. Advantageously, the monoalkoxysilane is chosen in the group consisting of:

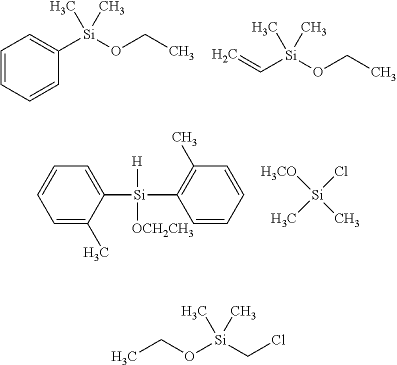

Advantageously, a separation step is provided after the reaction step, and possibly after the termination step. According to some embodiments, the separation step is performed by means of dialysis and/or ultrafiltration and/or dia-ultrafiltration.

According to some embodiments, the reaction step is performed in aqueous solution. Preferably, the solution has a pH lower than circa 5, advantageously higher than circa 0, or higher than circa 9, advantageously lower than circa 13.

Several organic solvents can be used in the mixing step. According to some embodiments, the organic solvent is selected in the group consisting of: methanol, chloroform, dichloromethane, tetrahydrofurane, acetonitrile, toluene, ethanol.

According to some embodiments, the active compound is selected in the group consisting of: 4-(Dicyanomethylene)-2-methyl-6-(4-dimethylaminostyryl)-4H-pyran, phthalocyanines, naphthalocyanines, carboxyimidic derivatives of perylene [for example, N,N′-Bis(2,5-di-tert-butylphenyl)-3,4,9,10-perylenedicarboximide], cyanines (CY7; CY5; CY3), complexes of Ir(III) (ECL), triethoxysilane derivatives of Rhodamine B, Fullerene C60 and its derivatives, organic lipophilic ECL active compounds (for example: rubrene, 9,10-diphenylanthracene, 9,10-dichloroanthracene, acridine, decacyclene, fluoranthene, etc.).

Advantageously, the active compound is an emitter compound. Advantageously, the active compound is luminescent or fluorescent.

According to some embodiments, the active compound is a photoluminescent one, that is to say a chemical species able to emit detectable electromagnetic radiations, advantageously with wavelengths from 200 nm to 1500 nm, advantageously higher than 500 nm, advantageously from 550 nm to 1500 nm.

Advantageously, the active compound is chosen among the group consisting of: cyanine (CY7; CY5; CY3), Ir(III) complexes and Ru(II) complexes. In some embodiments, the active compound is chosen among cyanine CY7 and cyanine CY5. According to some embodiments, the active compound is selected in the group consisting of: cyanine (CY7; CY5; CY3) and Ir(III) complexes. According to some specific embodiments, the active compound is an Ir(III) complex.

Cyanines are a family of luminescent compounds with a very wide structural variability.

In particular, closed chain cyanines have a structural formula that can be schematized as follows:

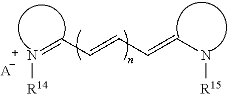

wherein A is advantageously Cl, Br, I, ClO₄. The two quaternary nitrogen atoms are inserted inside an eterocycle and are joint via a polymethinic chain; the polymethinic chain can be variously substituted.

The usual nomenclature distinguishes some subclasses that depend upon the number of the methinic groups present in the molecule.

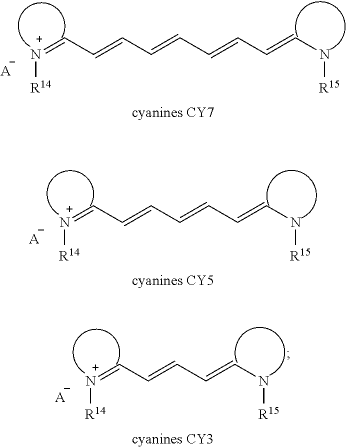

Both the nitrogen atoms can be independently part of an eteroaromatic ring, as for example pyrrole, imidazole, thiazole, pyridine, quinoline, benzothiazole, indole, benzo[e]indole, benzo[cd]indole etc. More precisely, some examples of eterocycles are: dimethylindole, benzodimethyilindole (which has 2 isomers, benzo[e] and benzo[cd]), benzozazoles, benzothiazoles, benzimidazoles. Particularly important are the examples including the eterocycles: 1,1-dimethyl-3-(methyl)-indole, 1,1-dimethyl-3-(ethyl)-indole, 1,1-dimethyl-3-(methyl)-benzo[e]indole, 1,1-dimethyl-3-(ethyl)-benzo[e]indole. In this case, the base structure can be schematized and rationalized as follows:

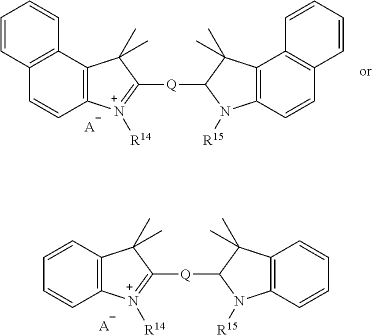

wherein A is advantageously Cl, Br, I, ClO₄, and the group Q is advantageously chosen among the group consisting of:

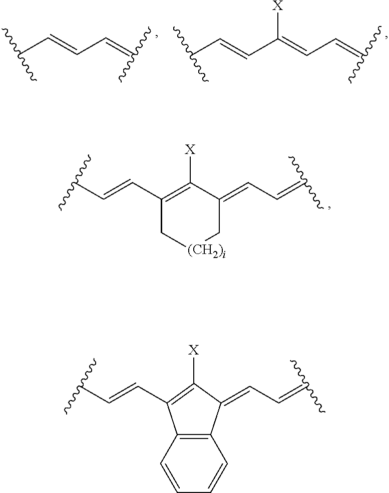

wherein the group X is chosen among the group consisting of F, Cl, Br, I,

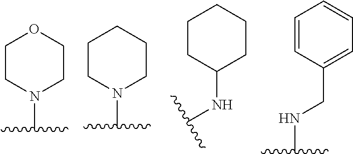

and wherein possibly i is 0 or 1.

And where the groups R¹⁴, R¹⁵ can be advantageously constituted, each one independently from the other, by an alkylic chain C₁-C₁₀.

An example of cyanine CY7 is the cyanine CY7ClIEt:

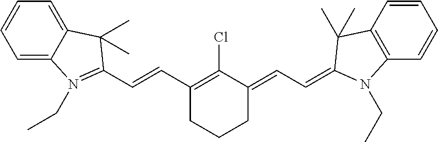

An example of cyanine CY5 is the cyanine Cy5BrNIEt:

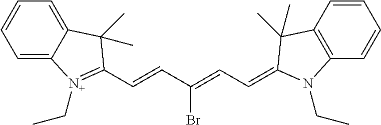

In some specific embodiments, the active compound is chosen among the group consisting of:

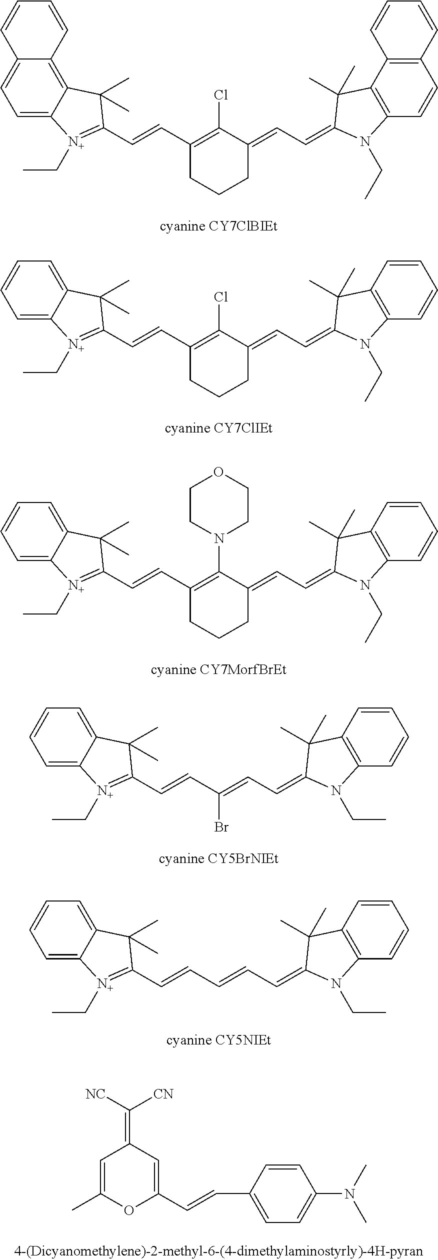

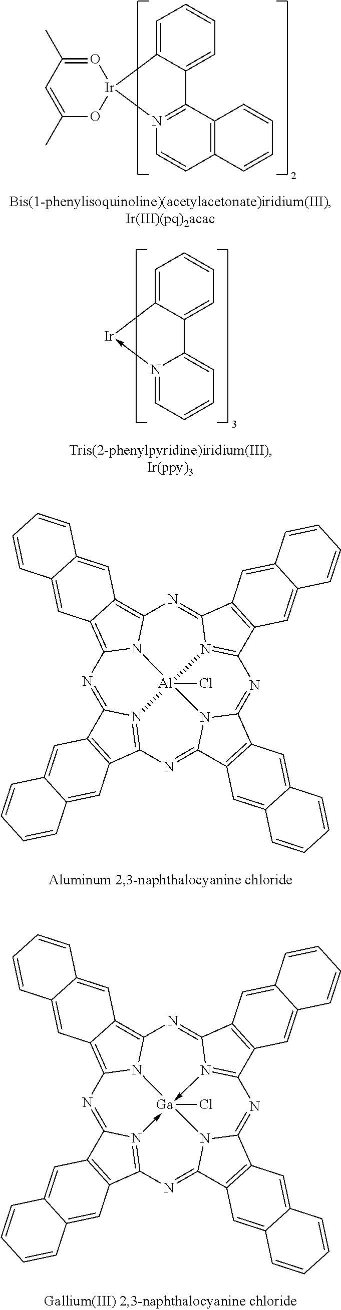

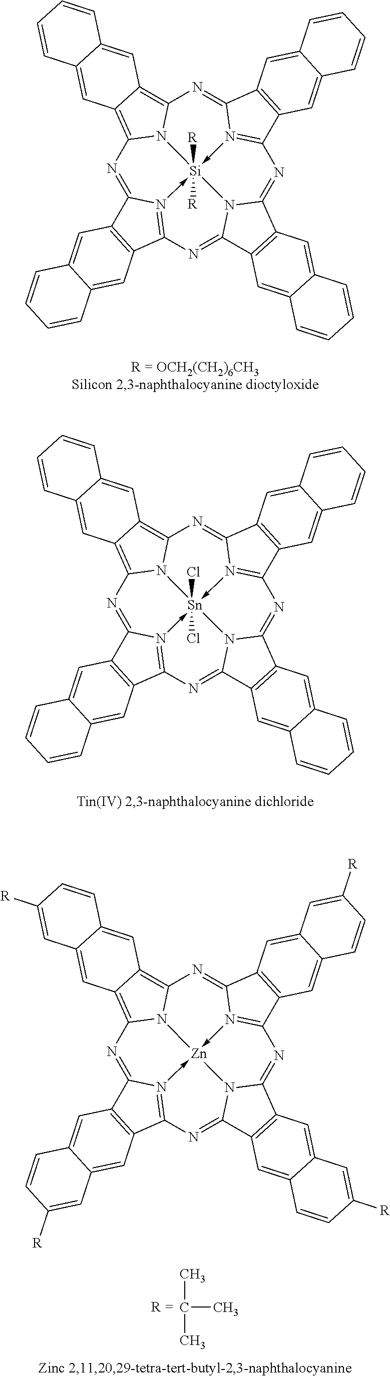

Experimentally, it has been observed that it is possible to improve the monodispersity (that is to say to reduce the amplitude of the distribution of the particle diameters) performing the reaction step in presence of a strong electrolyte. This is particularly useful when trialkoxysilanes is used instead of tetraalkoxysilanes and/or when the aqueous solution contains a weak acid (for example acetic acid) or a weak base (for example ammonia).

Therefore, advantageously, the aqueous solution in the reaction step includes the presence of a strong electrolyte (for example NaCl or KCl) with a concentration from circa 0.1M to circa 3.0M.

According to a second aspect of this invention, a particle realized in accordance with the method of the first aspect of the present invention is provided.

Advantageously, a particle obtained in accordance with the method of the first aspect of the present invention.

According to some embodiments, the particle has an average hydrodynamic diameter in water smaller than circa 100 nm, advantageously from circa 40 to circa 10 nm.

According to some embodiments, the core has a diameter lower than circa 30 nm, in particular from circa 5 to circa 15 nm.

The formation of the core, that can be produced by the hydrolysis and condensation processes of the organosilicates, yields to an efficient immobilization of the surfactant molecules in the particle.

The particles in accordance with the present invention can have the following applications:
as luminescent probes-labels (Zhao, X. Et al. J. Am. Chem. Soc. 2003, 125, (38), 11474-11475), of systems based on microarrays for diagnostic purposes (Wang, L. et al. Bioconjugate Chem. 2007, 18, (3), 610-613) and for in vivo imaging (Kobayashi, H. et al. Nano Lett. 2007, 7(6), 1711-1716) and in vitro imaging (Wang, L. et al. Bioconjugate Chem. 2007, 18, (2), 297-301);
for the development of magnetic particles with luminescent properties (Lu, C.-W. et al, Nano Lett. 2007, 7(1), 149-154; Lu, Y. et al. Nano Lett. 2002, 2(3), 183-186; Lattuada, M. et al, Langmuir 2007, 23, 2158-2168; Hu, F. et al, Biomacromolecules 2006, 7, 809-816; Yang, H.-H. et al, Anal. Chem. 2004, 76, 1316-1321; US 20070059705; US006545143B1).
for the photothermal therapy (PTT) (Everts, M.; Saini, V.; Leddon, J. L.; Kok, R. J.; Stoff-Khalili, M.; Preuss, M. A.; Millican, C. L.; Perkins, G.; Brown, J. M.; Bagaria, H.; Nikles, D. E.; Johnson, D. T.; Zharov, V. P.; Curiel, D. T. Nano Lett. 2006, 6(4), 587-591. Zharov, V. P.; Kim, J.-W.; Curiel, D. T.; Everts, M. Nanomedicine 2005, 1(4), 326-345);
for the photodynamic therapy (PDT). (McCaughan, J. S. Jr. Drugs and Aging 1999, 15, 46-68; Prasad, P. N. et al. Nano Lett., 2007, 7(9), 2835-2842; Prasad, P. N. et al. Proc. Natl. Acad. Sci. USA 2005, 102, 279-284; Prasad, P. N. et al. J. Am. Chem. Soc. 2003, 125, 7860-7865; US 20040180096; US 20060088599; US 20070217996);
for PET (positron emission tomography) applications. (Pressly, E. D.; Rossin, R.; Hagooly, A.; Fukukawa, K.-i.; Messmore, B. W.; Welch, M. J.; Wooley, K. L.; Lamm, M. S.; Hule, R. A.; Pochan, D. J.; Hawker, C. J. Biomacromolecules 2007, 8, (10), 3126-3134; R Cartier et al 2007, Nanotechnology 18 195102-195120).
for MRI (magnetic resonance imaging) imaging and of the contrast agents;
in ophthalmology as material used in the tissue welding obtained with the use of a laser (Chetoni, P. et al. J. Drug. Del. Sci. Tech., 2007, 17(1), 25-31).

According to further aspects of the present invention, it is herein provided what follows.

A particle in accordance with the second aspect of the present invention for diagnostic use. In particular, a particle in accordance with the second aspect of the present invention for diagnostic use in vivo.

A use of a particle in accordance with the second aspect of the present invention, for the production of a product for diagnostic use. In particular, a use of a particle in accordance with the second aspect of the present invention for the production of a product for diagnostic use in vivo.

A particle in accordance with the second aspect of the present invention to be used as a probe (label). A use as a probe (label) of a particle in accordance with the second aspect of the present invention.

A use of a particle in accordance with the second aspect of the present invention, for diagnostic purposes.

A diagnostic method that makes use of a particle in accordance with the second aspect of the present invention.

A particle in accordance with the second aspect of the present invention, for a therapeutic treatment, in particular for phototherapy.

A use of a particle in accordance with the second aspect of the present invention, for the production of a product to be used in a therapeutic treatment, in particular for phototherapic use.

A use of a particle in accordance with the second aspect of the present invention, for a therapeutic treatment, in particular phototherapy.

A therapeutic method, in particular a phototherapeutic one, that makes use of particles in accordance with the second aspect of the present invention.

By phototherapy it is meant photothermal therapy and/or photodynamic one; advantageously, photothermal.

The particles, depending on preparation, are compatible for all types of formulation and consequently of administration: in particular, for oral, parenteral or rectal administrations or for inhalations or insufflations (both through the mouth or through the nose). Formulations in view of parenteral administrations are favoured.

Formulations for injections can be in the form of unitdose, for example in vials or in multidose containers including preservatives. The dosage form can be a suspension, in aqueous or oily liquids, and can contain elements of the formulation such as dispersing and stabilizing agents.

The object of the present invention has, for example the following advantages with respect to the state of the art:

Technical Advantages:
ease of the synthetic procedures;
the obtained particles are sterically stabilized, monodispersed, and extremely stable, especially in aqueous solution and in physiological conditions of temperature, ionic strength and pH;
the particles are very soluble in aqueous environment;
it is possible obtain luminescent systems that emit in a wide range of wavelengths (UV-VIS-IR);
in the majority of the cases, the efficiency (luminescent quantum yield) of the active compounds (in particular, emissive ones) that are trapped or condensed inside the particles, increases;
there is an increase of the resistance to photodegradation of the active compounds that are inside the particles in comparison with the isolated active compound;
the particles can be functionalized with a great variety of functional groups on their surface;

### Economical Advantages:

the initial components and reagents necessary for the synthesis of the particles are extremely cheap;
in order to realize this kind of luminescent particles it is often possible to use commercial and cheap luminophores;
there is no need of special or expensive equipments in order to realize the invention;
the luminophore of election is introduced in the synthetic step, and it is quantitatively segregated in the core of the particle without wastes;

### Production Advantages:

the initial components and reagents necessary for the synthesis of the particles are easily available;
ease and fastness of the synthetic procedure for the preparation of the particles;
possible synthetic procedures for the modification of the surfactant Pluronic®F127 (or similar) are not laborious;
the possibility to use commercially available luminophores avoids the step of their synthesis or modification that are usually demanding, laborious and long procedures;
normal laboratory equipments are needed to realize the invention, the particle synthesis requires mild conditions of pressure and temperature;
the Pluronic® F127 (or similar) is a non toxic surfactant;
water is advantageously used as the reaction solvent.

The present patent application claims the priority of an Italian patent application (specifically, BO2008A000487), the content of which is herein entirely reported. In particular, the Italian patent application is herein incorporated by reference.

Further characteristics of the present invention will arise from the hereinafter description of some examples that are purely illustrative and not limiting.

### EXAMPLES

The UV-VIS absorption measurements have been performed using Perkin Elmer Lambda 650 and Lambda 45 spectrophotometers. The luminescence emission measurements have been performed using a Perkin Elmer LS50 spectrofluorimeter and a modular Edinburgh fluorimeter equipped with Picoquant lasers with different wavelengths and with polarizers and with a module for emission lifetime measurements.

The determinations of the hydrodynamic radius of the particles through DLS (dynamic light scattering) technique have been obtained with a NANO ZS by Malvern Instruments.

### Example 1

### Preparation of the Particles

A quantity of active compound in between 0.03E-6 and 8.00E-5 moles was mixed with 200 mg of surfactant (Pluronic® F 127).

To the mixture of the two solids a small quantity of dichloromethane (1-5 mL) was added in order to obtain an homogeneous solution of the surfactant and the active compound.

The organic solvent was then quantitatively evaporated under vacuum. To the obtained solid, 3130 mg of acidic aqueous solution were added (for example HCl 0.85M, alternatively it is possible to use also a basic solution) and stirred at room temperature. 336 mg (0.360 mL) of TEOS were added to the homogeneous obtained solution, and after 1 h and 45 min, 26 mg (0.030 mL) of DEDMS (dimethyldiethoxysilane) or of TMSCl (chlorotrimethylsilane) were added.

The reaction mixture was maintained under continuous stirring for another 48 hours.

Examples of lipophilic compounds that were used are: cyanines CY7 e CY5 (previously mentioned), 4-(Dicyanomethylene)-2-methyl-6-(4-dimethylaminostyryl)-4H-pyran, Bis(1-phenylisoquinoline) (acetylacetonate)iridium(III) (Ir(III)(pq)₂acac), Tris(2-phenylpyridine)iridium(III), Ir(ppy)₃, 9,10-diphenylanthracene, rubrene, Red Nile, naphthalocyanines (previously mentioned), N,N′-Bis(2,5-di-tert-butylphenyl)-3,4,9,10-perylenedicarboximide.

Hereafter one reports the relative characterizations and some non limiting examples.

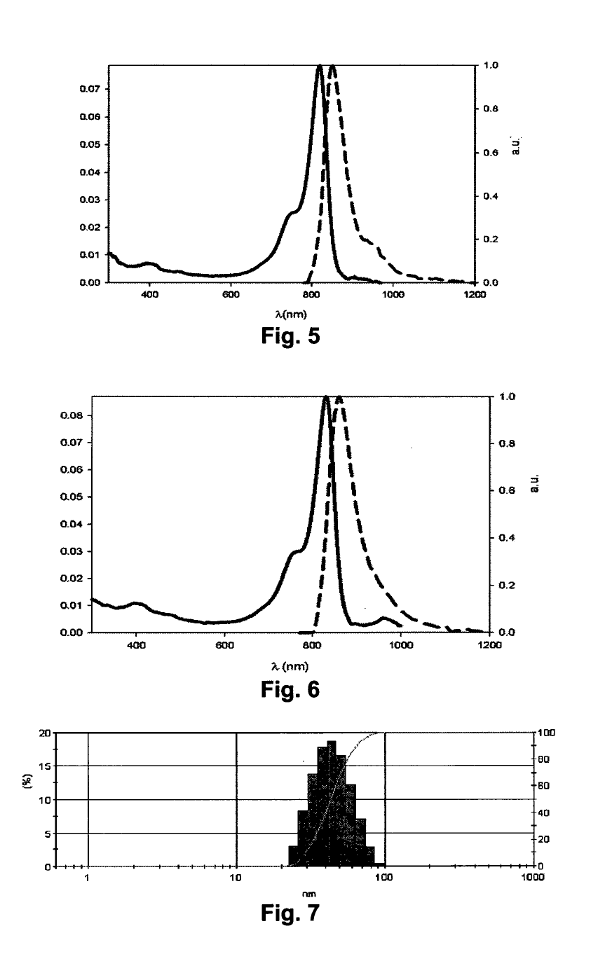

FIG. 5 shows the absorption spectrum (solid line) and the fluorescence emission spectrum (dotted line) of the cyanine CY7ClBIEt in dichloromethane.

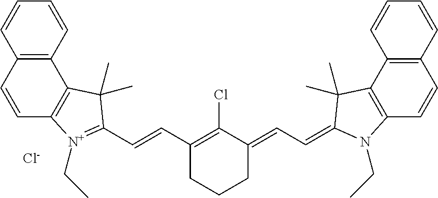

(λ_{exc}760 nm) (the wavelengths are reported on the x-axis; absorbance—on the left—and luminescence intensity—on the right—are reported on the y-axis) in EtOH.

FIG. 6 shows the absorption spectrum (solid line) and the fluorescence emission spectrum (dotted line) of particles containing the cyanine CY7ClBIEt (λ_{exc}=760 nm) (the wavelengths are reported on the x-axis; absorbance—on the left—and luminescence intensity—on the right—are reported on the y-axis) in H₂O.

FIG. 7 shows the dimensional distribution obtained via the DLS (dynamic light scattering) technique for particles containing the cyanine CY7ClBIEt in H₂O.

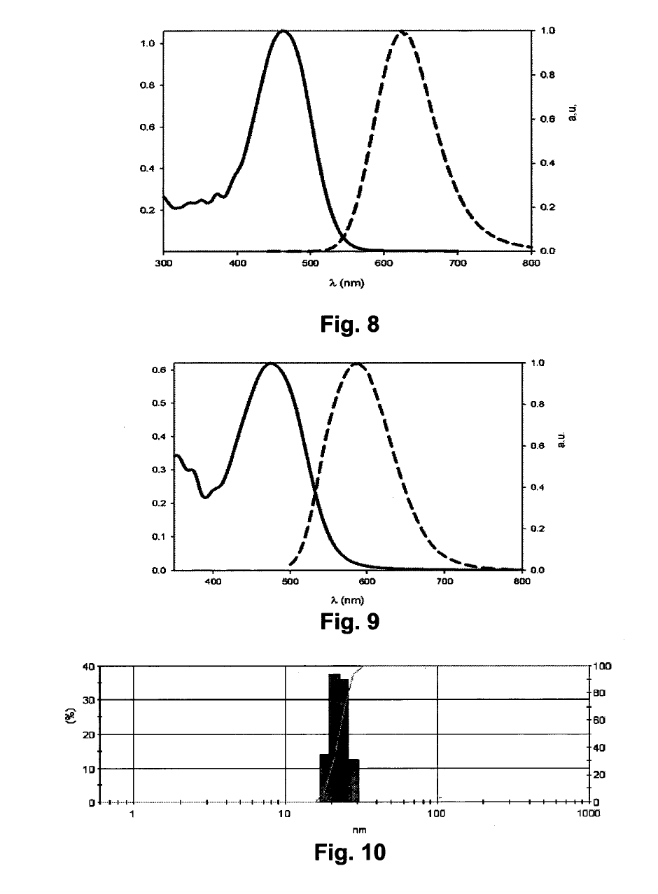

FIG. 8 shows the absorption spectrum (solid line) and the fluorescence emission spectrum (dotted line) of the 4-(Dicyanomethylene)-2-methyl-6-(4-dimethylaminostyryl)-4H-pyran

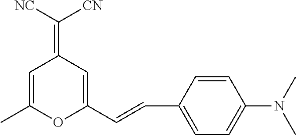

(the wavelengths are reported on the x-axis; absorbance—on the left—and luminescence intensity—on the right—are reported on the y-axis) in acetonitrile.

FIG. 9 shows the absorption spectrum (solid line) and the fluorescence emission spectrum (dotted line) of particles containing 4-(Dicyanomethylene)-2-methyl-6-(4-dimethylaminostyryl)-4H-pyran (the wavelengths are reported on the x-axis; absorbance—on the left—and luminescence intensity—on the right—are reported on the y-axis) in H₂O.

FIG. 10 shows the dimensional distribution obtained via the DLS (dynamic light scattering) technique for particles containing the 4-(Dicyanomethylene)-2-methyl-6-(4-dimethylaminostyryl)-4H-pyran in H₂O.

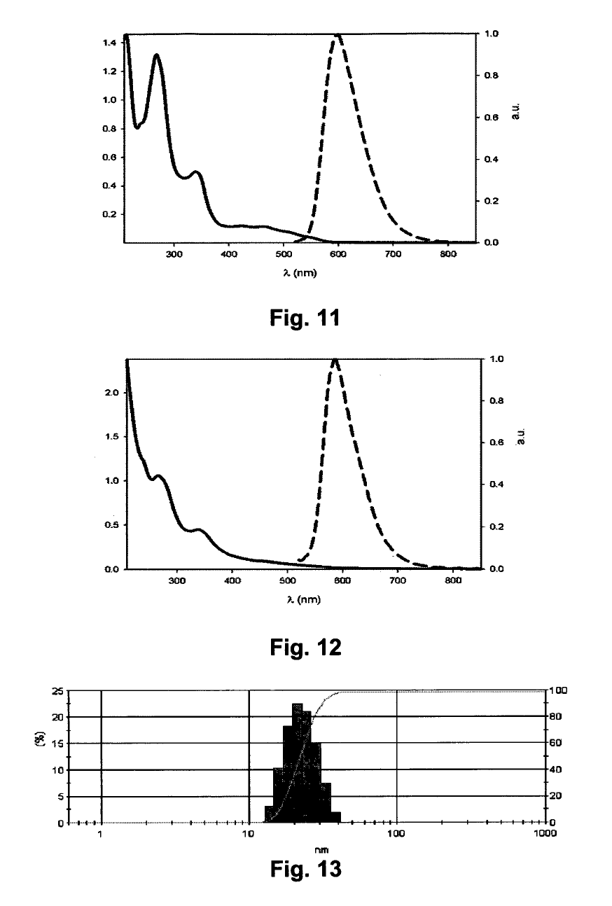

FIG. 11 shows the absorption spectrum (solid line) and the fluorescence emission spectrum (dotted line) of the Bis(1-phenylisoquinoline) (acetylacetonate)iridium(III) (Ir(III)(pq)₂acac)

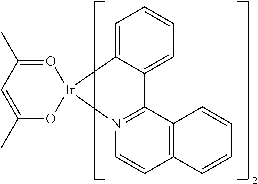

(the wavelengths are reported on the x-axis; absorbance—on the left—and luminescence intensity—on the right—are reported on the y-axis) in EtOH.

FIG. 12 shows the absorption spectrum (solid line) and the fluorescence emission spectrum (dotted line) of particles containing Bis(1-phenylisoquinoline) (acetylacetonate)iridium(III) (Ir(III)(pq)₂acac) (the wavelengths are reported on the x-axis; absorbance—on the left—and luminescence intensity—on the right—are reported on the y-axis) in H₂O.

FIG. 13 shows the dimensional distribution obtained via the DLS (dynamic light scattering) technique for particles containing the Bis(1-phenylisoquinoline) (acetylacetonate)iridium(III) (Ir(III)(pq)₂acac) in H₂O.

### Example 2

### Synthesis of 8-Oxo-3-propylaminotriethoxysilyl-8H-acenaphtho[1,2-b]pyrrol-9-carbonitrile

The synthesis of the compound was obtained starting from precursor 1, whose synthesis, as well as the synthesis of the model compound 8-Oxo-3-propylamino-8H-acenaphtho[1,2-b]pyrrol-9-carbonitrile, was described by Xiao Y. et al. in Chem. Commun., 2005, 239.

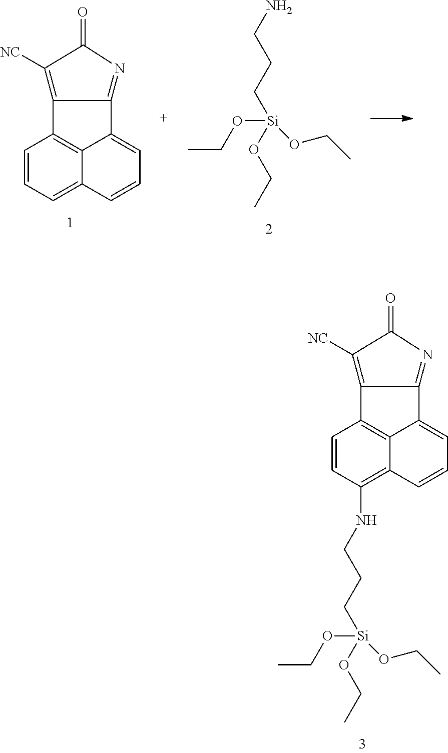

A solution of (3-aminopropyl)triethoxysilane (5.07 mL, 21.72 mmol) in 30 mL of acetonitrile was added at room temperature to a suspension of 1 (1.00 gr, 4.3 mmol) in 100 mL of the same solvent. The reaction mixture changed colour from yellow-brown to deep red (TLC: dichloromethane and dichloromethane/ethanol 10/0.2). The solvent was removed under reduced pressure and the residue was solubilized with a small quantity of diethyl ether. Then, petroleum ether was added (about 400 mL) and the resulting suspension was filtered. The solid was then purified by flash chromatography on silica gel (dichloromethane/ethanol 10/0.2) obtaining 330 mg of 8-oxo-3-propylaminotriethoxysilyl-8H-acenaphtho[1,2-b]pyrrol-9-carbonitrile (3, yield 15%) as a yellow-golden solid.

¹H NMR (250 MHz, CDCl3) δ: 0.78 (t, 2H, J=8.1 Hz), 1.15 (t, 9H, J=7.3 Hz), 2.01 (m, 2H), 3.76 (q, 6H, J=7.3 Hz), 4.07 (q, J=7.3 Hz), 7.58-7.71 (m, 3H), 8.04 (t, 2H, J=2.6 Hz), 8.35 (d, 1H, J=9.5 Hz), 8.45-8.49 (dd, 1H, 3J=7.0 Hz, 4J=0.9 Hz).

¹³C NMR (62.9 MHz, CDCl3, 25° C.) δ: 7.6, 18.7, 23.9, 47.6, 58.9, 84.9, 118.8, 123.2, 125.6, 126.4, 127.3, 127.5, 127.8, 129.5, 132.6, 134.3.

ESI-MS, m/z (M+H) 450.2.

### Example 3

### Preparation of Particles

8-Oxo-3-propylaminotriethoxysilyl-8H-acenaphtho[1,2-b]pyrrol-9-carbonitrile (see example 2) was used as the active compound. The procedure described in example 1 was followed.

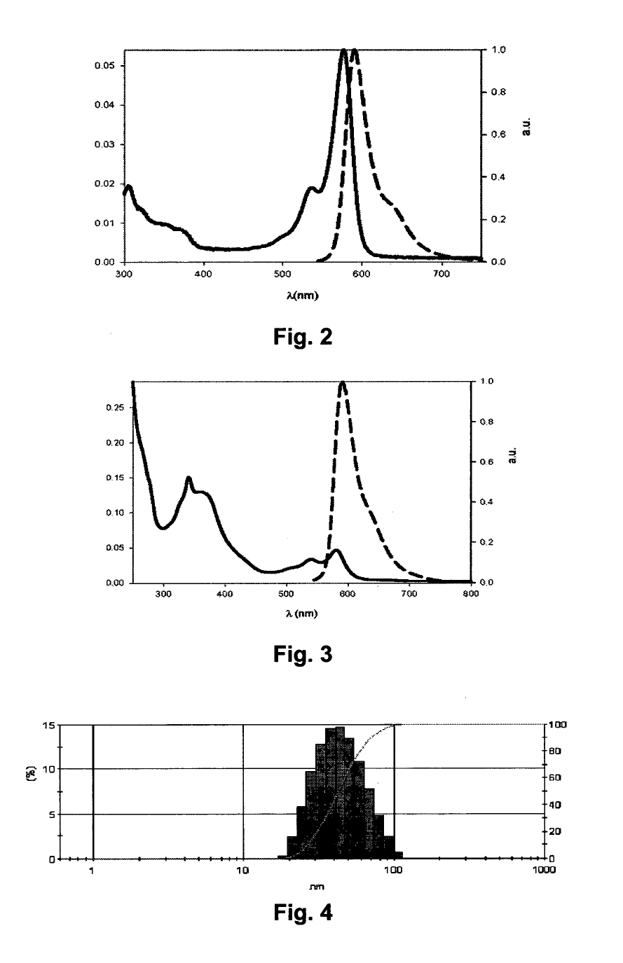

FIG. 2 shows the absorption (solid line) and emission (dotted line) spectrum of 8-Oxo-3-propylaminotriethoxysilyl-8H-acenaphtho[1,2-b]pyrrol-9-carbonitrile (λ_{exc}=535 nm) (wavelengths are reported on the x-axis, while absorbance—on the left—and luminescence intensity—on the right—are reported on the y-axis) in EtOH.

FIG. 3 shows the absorption (solid line) and emission (dotted line) spectrum of particles containing 8-Oxo-3-propylaminotriethoxysilyl-8H-acenaphtho[1,2-b]pyrrol-9-carbonitrile (λ_{exc}=535 nm) (wavelengths are reported on the x-axis, while absorbance—on the left—and luminescence intensity—on the right—are reported on the y-axis) in H₂O.

FIG. 4 shows the dimensional distribution of particles containing 8-Oxo-3-propylaminotriethoxysilyl-8H-acenaphtho[1,2-b]pyrrol-9-carbonitrile obtained by means of DLS (dynamic light scattering) technique in water.

### Example 4

### Preparation of Particles

Fluorescein sodium salt was used as the active compound.

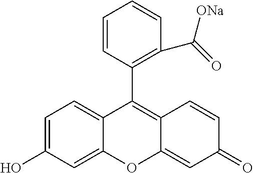

The procedure described in the supporting information of the article J. Am. Chem. Soc. 2006, 128, 6447-6453 (paragraph 2.4, page S4) was followed.

### Example 5

### Preparation of Particles

Red Nile was used as the active compound.

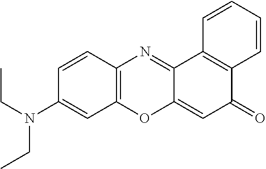

The procedure described in the supporting information of the article J. Am. Chem. Soc. 2006, 128, 6447-6453 (paragraph 2.4, page S4) was followed.

### Example 6

### Leaching Tests

Reaction mixtures of example 1 (with fluorescein sodium salt, with methanol as the organic solvent) and 4 were diluted to 50 mL with Milli-Q water and subjected to dia-ultrafiltration (regenerated cellulose membrane, cut-off 10 Kda, diameter 47 mm, Millipore cell of 75 mL, P=0.5 atm N₂, flux of diffusate about 0.25 mL/min, volume of diffusate 3000 mL, pH 7,2).

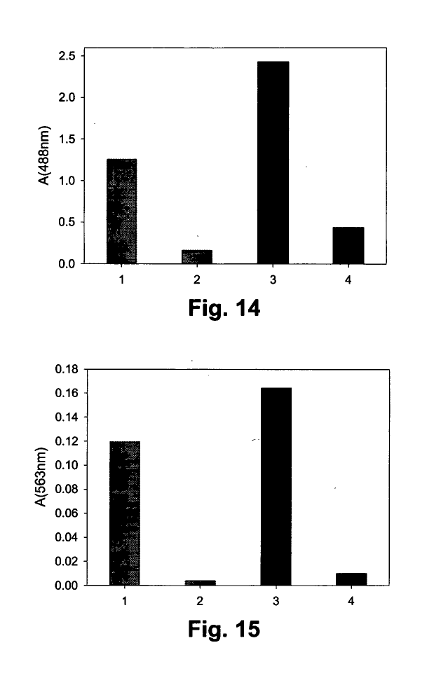

The absorbance of the particles of example 4, before and after ultrafiltration (column 1 and 2) and of example 1 before and after ultrafiltration (column 3 and 4) were evaluated. The results are shown in FIG. 14 (the absorbance at 488 nm is reported on the y-axis). As can be noticed, the amount of active compound which remains inside the particles after a prolonged dia-ultrafiltration treatment, is different in the two cases. The methodology illustrated in example 1 allows to keep approximately a double quantity of active compound inside the nanoparticles.

Besides, one can notice that the methodology illustrated in example 1 requires far less time with respect to the methodology in example 4.

### Example 7

### Leaching Tests

Reaction mixtures of example 1 (with Red Nile) and 5 were diluted to 50 mL with Milli-Q water and subjected to dia-ultrafiltration (regenerated cellulose membrane, cut-off 10 Kda, dialysis solution PBS 1×pH 7,2).

The absorbance of the particles of example 5, before and after ultrafiltration (column 1 and 2) and of example 1 before and after ultrafiltration (column 3 and 4) were evaluated. The results are shown in FIG. 15 (the absorbance at 563 nm is reported on the y-axis). As can be noticed, the amount of active compound which remains inside the particles after a prolonged dia-ultrafiltration treatment, is different in the two cases. The methodology illustrated in example 1 allows to keep approximately a double quantity of active compound inside the nanoparticles.

Besides, one can notice that the methodology illustrated in example 1 requires far less time with respect to the methodology in example 5.

### Example 8

### Tests of In Vivo Imaging

Particles that can be used in this kind of experiments must have absorption and emission wavelengths in a region of the electromagnetic spectrum where tissues are transparent to light radiations (λ≧650-700 nm). For this reason, the utilization of particles containing active compounds like cyanines CY7 and CY5, which absorb and emit in the infrared region, is particularly advantageous.

Particles obtained in agreement with example 1 containing the cyanine CY7ClBIEt (average total diameter 30 nm), have been used for the following tests. For these applications, samples were subjected to dialysis and/or ultrafiltration and conveniently diluted with a PBS 10× buffer in order to reach a pH value of 7,2.

Images were acquired on nude athymic nu/nu female mice (HSD Athymic Nude Fox1 nu-homozygotes) from 3 to 4 weeks old. Solutions of particles comprising cyanine CY7ClBIEt were used in the experiments with a dosage of 0.005-0.010 ml per g of body weight of the animal (200 μL, approximate concentration of particles: 2×10⁻⁷M per litre of physiological buffer PBS 1× at pH 7,2).

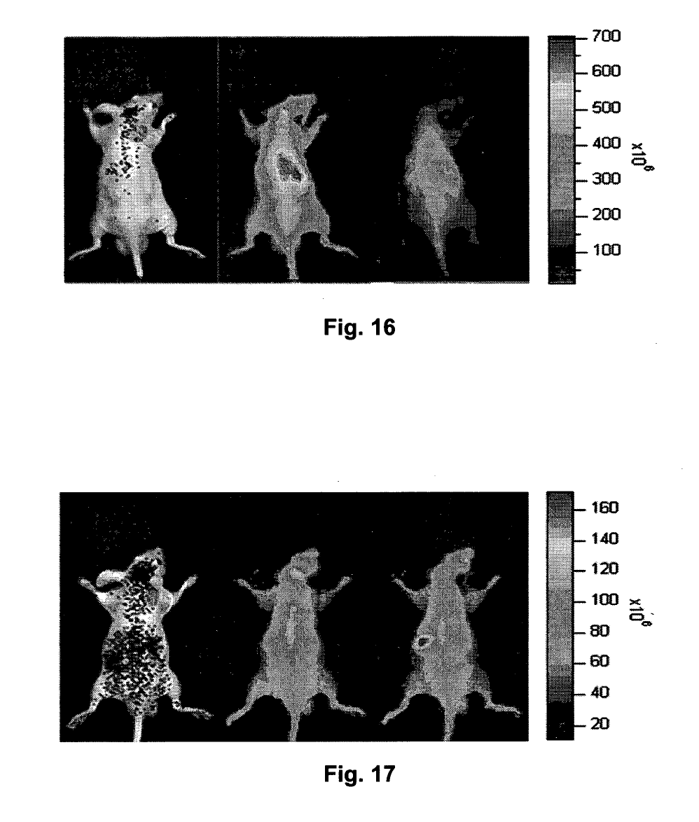

In FIG. 16, one can notice the good intensity of the luminescence signal and its quite uniform distribution in the organism, with accumulation areas in some organs (liver). On the left is reported the image before the inoculation, in the middle the image right after the inoculation, and on the right the image 3 hours and 20 minutes after the inoculation (images were acquired with exc./em.—ICG/ICG (Indocyanine Green) filters).

For comparison, images obtained in similar conditions were also acquired with a commercial product, which is commonly used for this kind of experiments, that is Quantum Dots 800 (QDs 800), sold by Invitrogen®.

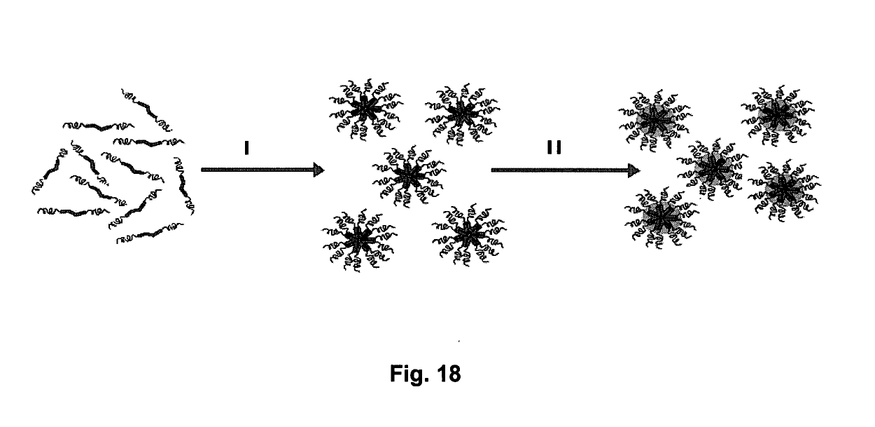

The image shown in FIG. 17 was acquired injecting a 2004, sample with a concentration of about 4×10⁻⁷M QDs 800 per litre. On the left is reported the image before the inoculation, in the middle the image right after the inoculation, and on the right the image 3 hours and 20 minutes after the inoculation (images were acquired with exc./em.—ICG/ICG (Indocyanine Green) filters).

Next to the series of images acquired during each experiment is shown a luminescence intensity scale in pseudo-colours. The recorded intensities in the images obtained with our samples, also considering the lower concentration of luminescent particles inoculated, are much greater in comparison with the ones recorded in the experiment with QDs 800.

1. Method for the preparation of an active particle comprising a mixing step, during which at least a substantially lipophilic active compound is mixed with a plurality of molecules of at least a surfactant in an organic solvent; an evaporation step, which follows the mixing step, and during which the organic solvent is evaporated in order to obtain a residue; a reaction step, which follows the evaporation step, and during which a plurality of molecules of at least an alkoxysilane are mixed with the residue and silanized in the presence of water and of the residue; the alkoxysilane being chosen between a tetraalkoxysilane and a trialkoxysilane; the surfactant comprising the following structure:
Hydro¹-Lipo-Hydro²
wherein Lipo represents a substantially hydrophobic chain; Hydro¹ and Hydro² each representing a respective substantially hydrophilic chain.

2. Method according to claim 1, wherein the reaction step takes place in an aqueous solution whose pH is lower than approximately 5 or higher than approximately 9.

3. Method according to claim 1, wherein Hydro¹ represents a chain 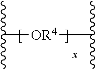
wherein x is from 40 to 130 and R⁴ is a linear C₁-C₃ alkyl group; Hydro² represents a chain 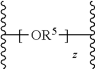
wherein z is from 40 to 130 and R⁵ is a linear C₁-C₃ alkyl group; Lipo represents a chain 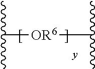
wherein y is from 20 to 85, R⁶ is a branched C₃-C₄ alkyl group; y is lower than or equal to x and z; said alkoxysilane has a formula chosen in the group consisting of: 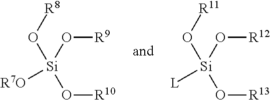
wherein R⁷, R⁸, R⁹, R¹⁰, R¹¹, R¹² and R¹³ are, independently of each other, a C₁-C₄ alkyl group; L represents a substantially lipophilic molecular portion.

4. Method according to claim 1, wherein the alkoxysilane has the formula 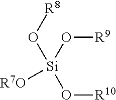
wherein R⁷, R⁸, R⁹ and R¹⁰ are, independently of each other, a C₁-C₂ alkyl group; R¹, R², R³ represent, independently of each other, a C₁-C₂) alkyl group; Hydro¹ represents a chain 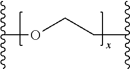
wherein x is from 80 to 120; Hydro² represents a chain 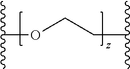
wherein z is from 80 to 120; Lipo represents a chain 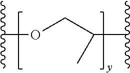
wherein y is from 50 to 80.

5. Method according to claim 1, wherein the reaction step takes place in a solution; at the beginning of the reaction step the molar ratio of the active compound and the alkoxysilane is from 0.002% to 5%, in particular from 0.01% to 0.5%; the molar ratio of alkoxysilane and surfactant is lower than approximately 110.

6. Method according to claim 1, wherein the duration of the reaction step is lower than approximately six hours.

7. Method according to claim 1, further comprising a purification step following the reaction step.

8. Method according to claim 1, further comprising a termination step, during which the reaction step is terminated by means of the addition of a termination compound chosen in the group consisting of:
monoalkoxysilane, dialkoxysilane, monohalosilane, dihalosilane; in particular, the termination step follows the reaction step and precedes the purification step.

9. Method according to claim 8, wherein the reaction step takes place in an aqueous solution whose pH is lower than approximately 5 or higher than approximately 9; the pH is higher than approximately 0 and lower than approximately 13; the termination compound is chosen between: a dialkoxysilane, in particular diethoxydimethylsilane, and a monohalosilane, in particular chlorotrimethylsilane.

10. Method according to claim 1, wherein the active compound is an emitting compound.

11. Method according to any claim 1, wherein the active compound is chosen in the group consisting of: CY5 and CY7 cyanines, Ru(II) and Ir(III) complexes.

12. Particle obtainable by the method according to claim 1.

13. Particle according to claim 12, wherein the surfactant has an average molecular weight of at least 6 KDa; the ratios between the Lipo average molecular weight and the Hydro¹ average molecular weight and between the Lipo average molecular weight and the Hydro² average molecular weight are, independently of each other, from approximately 0.4 to approximately 2.0.

14. Particle according to claim 12, having an average hydrodynamic diameter in water smaller than approximately 100 nm, in particular from approximately 40 to approximately 10 nm.

15. Particle according to claim 12 for diagnostic use in vivo.

16. Use of a particle according to claim 12, for the production of a product for diagnostic use, more particularly in vivo.

17. Use of a particle according to claim 12, as a probe.

18. Particle according to claim 12 for a therapeutic treatment.

19. Use of a particle according to claim 12 for the production of a product for phototherapeutic use.

20. Pharmaceutical preparation comprising a particle according to claim 12.
